(19) United States
(12) Patent Application Publication
(10) Pub. No.: US 2024/0197805 A1
(43) Pub. Date: Jun. 20, 2024

(54) COMPOSITE FOR RELIEVING FUNCTIONAL DYSPEPSIA, PREPARATION METHOD THEREFOR, USE THEREOF, DRUG, FOOD AND HEALTH-CARE PRODUCT

(71) Applicant: INFINITUS ( CHINA ) COMPANY LTD., Guangdong (CN)

(72) Inventors: Yaqian GAO, Guangdong (CN); Na ZHAO, Guangdong (CN); Jian TANG, Guangdong (CN); Hongying LI, Guangdong (CN); Yiting Yang, Guangdong (CN); Ya Cai, Guangdong (CN); Xiaochun XIAO, Guangdong (CN)

(73) Assignee: INFINITUS ( CHINA ) COMPANY LTD., Guangdong (CN)

(21) Appl. No.: 18/297,644

(22) Filed: Apr. 10, 2023

(30) Foreign Application Priority Data
Dec. 15, 2022 (CN) 202211619952.2

(51) Int. Cl. A61K 36/752; A23L 33/105; A61K 36/60

(52) U.S. Cl. CPC A61K 36/752; A23L 33/105; A61K 36/60; A61K 2236/39; A61K 2236/51

(57) ABSTRACT

The present invention provides a composite for relieving functional dyspepsia, a preparation method therefor, a use thereof, a drug, a food and a health-care product. The composite comprises the following components in parts by weight: 2 to 8 parts of dried orange peels, 1 to 6 parts of figs, and 1 to 5 parts of seville orange flowers. The composite of the present invention is prepared from medicinally and edibly homologous natural plant raw materials, which has good safety without side effects, and can be used to treat and/or prevent functional dyspepsia. The composite can also be used to prevent depression and improve sleep, with significant, mild and gradual curative effects, so as to provide a patient with daily health care and drug security.

### CROSS-REFERENCE TO RELATED APPLICATION

This application claims the priority benefit of China application serial no. 202211619952.2, filed on Dec. 15, 2022. The entirety of the above-mentioned patent application is hereby incorporated by reference herein and made a part of this specification.

### BACKGROUND

### Technical Field

The present invention belongs to relates to the technical field of medicine and food. More specifically, the present invention relates to a composite for relieving functional dyspepsia and a preparation method therefor.

### Description of Related Art

Functional dyspepsia (FD) is a gastroenteropathy and usually accompanied by symptoms such as nausea, vomiting and the like, can occur repeatedly, and has a disease course exceeding one month. Besides, a patient with FD may have obvious symptoms such as early satiety, abdominal discomfort, anorexia, intermittent eructation and the like. Functional dyspepsia has no organic lesions, but is chronic, recurrent and difficult to relieve, and may affect life quality of the patient. In recent years, with the development of economy, the life quality of people is improved day by day and a dietary structure is diversified gradually, such that an incidence rate of functional dyspepsia is increased year by year.

At present, the academic community considers that the occurrence of functional dyspepsia is related to gastric origin factors such as gastric motility disorder, gastric paresthesia, gastric electrical dysrhythmia, Helicobacter pylori (Hp) and the like, and factors such as diet, life style, social psychology and the like. Particularly, factors such as a brain-gut axis and gastrointestinal hormones and the like can play a more important role in the functional dyspepsia. In the traditional Chinese medicine field, research on treatment of functional dyspepsia has a long history and rich clinical experience. The disease is mostly treated with symptoms of distention and fullness and spleen deficiency, which has certain advantages and characteristics. Doctors in different generations believe that functional dyspepsia is fullness mostly caused by food stagnation and obstruction or liver qi stagnation, and further believe that long-term effects of diet, emotion, fatigue and the like can cause weakness of the spleen and the stomach and malnutrition of the stomach, resulting in distention and fullness. Therefore, the functional dyspepsia is traditionally mostly treated by needling Zusanli, Zhongwan and Neiguan to soothing the liver, lowering the adverse qi, strengthening the spleen and nourishing the stomach. However, the modern medicine more recognizes a physiological-psychological-social disease mode and considers that stress reaction and emotional changes of a patient caused by internal and external environment changes can cause spleen deficiency (gastrointestinal motility, absorption, hormone secretion abnormality, immune disorder and the like) through liver depression (neurotransmitter, hormone and function changes), which is actually also a relation between brain-gut axes.

The prior art provides a medicine for treating functional dyspepsia. The medicine comprises round cardamom fruits, lignum sappan, pepper, rhizoma picrorhizae, sea cucumber, Breynia fruticosa, Umbilicaria hypococcinea, exocarpium citrus grandis, puberulous glochidion herb, sea buckthorn, Embelia laeta, blood clam, Chinese fir, Ixeris chinensis, semen aesculi and rhizoma nelumbinis. However, the medicine contains up to 16 traditional Chinese medicines and the rare traditional Chinese medicines such as sea cucumber and the like, has high cost and is difficult to popularize and use. Therefore, it is particularly important to develop a low-price and high-efficiency medicine capable of relieving functional dyspepsia.

### SUMMARY

Aiming at the defects of the prior art, the present invention provides a composite for relieving functional dyspepsia, which is prepared from medicinally and edibly homologous plant raw materials with a low cost, a good curative effect and a small side effect. The composite is suitable for long-term administration, can be used as a daily health-care product, and provides a new choice for treating functional dyspepsia.

A first objective of the present invention is to provide a composite for relieving functional dyspepsia.

A second objective of the present invention is to provide a preparation method for a composite for relieving functional dyspepsia.

A third objective of the present invention is to provide use of the composite in preparation of a drug, a food and/or a health-care product for treating and/or preventing functional dyspepsia.

A fourth objective of the present invention is to provide a drug, a food and/or a health-care product for treating and/or preventing functional dyspepsia.

The foregoing objectives of the present invention are realized by the following technical solution:

The present invention provides a composite for relieving functional dyspepsia, wherein

the composite comprises the following components in parts by weight: 2 to 8 parts of dried orange peels, 1 to 6 parts of figs, and 1 to 5 parts of seville orange flowers.

Preferably, the composite comprises the following components in parts by weight: 4 to 6 parts of dried orange peels, 2 to 4 parts of figs, and 1 to 3 parts of seville orange flowers. Most preferably, the composite comprises the following components in parts by weight:

5 parts of dried orange peels, 3 parts of figs, and 2 parts of seville orange flowers.

The present invention further provides a preparation method for a composite for relieving functional dyspepsia. The method comprises uniformly mixing dried orange peels, figs and seville orange flowers of formula amount to obtain a mixture, and successively subjecting the obtained mixture to crushing, water extraction, filtration, centrifugation, concentration and freeze-drying, so as to obtain the composite.

Preferably, the water extraction is as follows: performing heating at 95° C. to 100° C. for 2 to 3 hours.

Further preferably, the water extraction is performed step by step. The step-by-step water extraction can enable the extraction to be more sufficient and a yield of an effective ingredient to be higher.

Further preferably, the step-by-step water extraction is as follows: adding water for the first time into the crushed traditional Chinese medicine mixture, performing heating at 95° C. to 100° C. for 1.2 to 1.8 hours (most preferably, for 1.5 hours), and then adding water for the second time, and performing heating at 95° C. to 100° C. for 0.8 to 1.2 hours (most preferably, for 1 hour).

Further preferably, a ratio of the amount of water added for the first time to the use amount of the crushed traditional Chinese medicine mixture is (9 to 11) mL:1 g, most preferably, 10 mL:1 g.

Further preferably, a ratio of the amount of water added for the second time to the use amount of the crushed traditional Chinese medicine mixture is (7 to 9) mL:1 g, most preferably, 8 mL:1 g.

Preferably, the filtration is performed using a 30 to 50-mesh gauze. Most preferably, the 40-mesh gauze is used.

Preferably, the centrifugation is performed at 4,000 to 6,000 rpm for 5 to 8 minutes. Most preferably, the centrifugation is performed at 5,000 rpm for 6 minutes.

Preferably, the concentration is performed at a temperature of 55 to 65° C. and a pressure intensity of −0.1 MPa to −0.08 MPa until a brix value is 20% to 30%. Most preferably, the concentration is performed at a temperature of 60° C. and a pressure intensity of −0.09 MPa until a brix value is 25%.

Preferably, the freeze-drying is performed at a temperature of −50° C. to −40° C. and a pressure intensity of 10 to 13 Pa for 4 to 6 hours. Most preferably, the freeze-drying is performed at a temperature of −45° C. and a pressure intensity of 12 Pa for 5 hours.

The composite of the present invention may be used for treating and/or preventing functional dyspepsia, may further be used for preventing depression and improving sleep, and is suitable to be prepared into a drug, a food and/or a health-care product for treating and/or preventing functional dyspepsia. Therefore, the composite is used in preparation of a drug, a food and/or a health-care product for treating and/or preventing functional dyspepsia, and the drug, the food and/or the health-care product for treating and/or preventing functional dyspepsia containing the composite should be further within the protection scope of the present invention.

Preferably, the drug may further comprise a pharmaceutically acceptable excipient and is prepared into different dosage forms, such as a chewable tablet, a solid granule, a straight drink powder or a liquid strip bag, etc. When the dosage form is a chewable tablet, the excipient comprises isomaltitol, maltodextrin, magnesium stearate and stevioside, and preferably, a mass ratio of the composite, isomaltitol, maltodextrin, magnesium stearate and stevioside is (20 to 35):(35 to 55):(15 to 25):(1 to 1.5):(0.01 to 0.05). When the dosage form is a solid granule, the excipient comprises maltodextrin and corn syrup, and preferably, a mass ratio of the composite, maltodextrin and corn syrup is (80 to 90):(7 to 15):(7 to 15). When the dosage form is a straight drinking powder, the excipient comprises xylitol and erythritol, and preferably, a mass ratio of the composite, xylitol and erythritol is (65 to 75):(12.5 to 17.5):(12.5 to 17.5).

The present invention has the following beneficial effects:
1. The composite provided by the present invention is prepared from medicinally and edibly homologous plant raw materials, which has good safety without side effect, has excellent effects of promoting gastric motility, correcting brain-gut axis dysfunction and inhibiting inflammatory reaction, and is used for treating and/or preventing functional dyspepsia. The composite may further be used for preventing depression and improving sleep with significant, mild and gradual curative effects, and is suitable for being prepared into a drug, a food and/or a health-care product for treating and/or preventing functional dyspepsia, depression and insomnia.
2. The present invention performs process innovation and improvement on preparation of the composite. The process is clean and environment-friendly, has high safety, and maintains flavor and effective components of the raw materials to the maximum extent. The composite may be prepared into various different dosage forms, further improves compliance of a patient with functional dyspepsia when eating, and provides the patient with daily health care and drug security.

### BRIEF DESCRIPTION OF THE DRAWINGS

FIG. 1 shows results of an intestinal propulsive rate and a stomach residue rate.

FIG. 2 shows a change of content of motilin.

FIG. 3 shows a change of content of cholecystokinin.

FIG. 4 shows a change of content of vasoactive intestinal peptide.

FIG. 5 shows a change of content of TNF-α.

FIG. 6 shows a change of content of IFN-γ.

FIG. 7 shows a change of content of c-kit.

FIG. 8 shows results of a tail suspension test and a forced swim test.

### DESCRIPTION OF THE EMBODIMENTS

The present invention is further described with reference to the drawings and specific examples, which are not intended to limit the present invention in any form. Unless otherwise specified, the reagents, methods and equipment used in the present invention are conventional in the art.

Unless otherwise specified, the reagents and materials used in the examples are commercially available.

### Example 1 Composite for Relieving Functional Dyspepsia

5 g of dried orange peels, 3 g of figs, and 2 g of seville orange flowers were uniformly mixed to obtain a mixture. The obtained mixture was crushed, then 100 ml of water was added, and the mixture was decocted at 100° C. for 1.5 hours. Then 80 mL of water was added, and the mixture was decocted at 100° C. for 1 hour. Then the mixture was filtered by using a 40-mesh gauze, centrifuged at 5,000 rpm for 6 minute, concentrated at a temperature of 60° C. and a pressure intensity of −0.09 MPa until a brix value was 25%, and dried at a temperature of −45° C. and a pressure intensity of 12 Pa for 5 hours to obtain the composite.

### Example 2 Composite for Relieving Functional Dyspepsia

3 g of dried orange peels, 1 g of figs, and 0.5 g of seville orange flowers were uniformly mixed to obtain a mixture. The obtained mixture was crushed, then 40.5 mL of water was added, and the mixture was decocted at 95ºC for 1.2 hours. Then 31.5 mL of water was added, and the mixture was decocted at 95° C. for 1.2 hours. Then the mixture was filtered by using a 50-mesh gauze, centrifuged at 4,000 rpm for 8 minutes, concentrated at a temperature of 55° C. and a pressure intensity of −0.1 MPa until a brix value was 20%, and dried at a temperature of −50° C. and a pressure intensity of 10 Pa for 4 hours to obtain the composite.

### Example 3 Composite for Relieving Functional Dyspepsia

4 g of dried orange peels, 4 g of figs, and 3 g of seville orange flowers were uniformly mixed to obtain a mixture. The obtained mixture was crushed, 121 mL of water was added, the mixture was decocted at 100° C. for 1.8 hours. Then 99 mL of water was added, the mixture was decocted at 100° C. for 0.8 hour. Then the mixture was filtered by using a 30-mesh gauze, centrifuged at 6,000 rpm for 5 minutes, concentrated at a temperature of 65° C. and a pressure intensity of −0.08 MPa until a brix value was 30%, and dried at a temperature of −40° C. and a pressure intensity of 13 Pa for 6 hours to obtain the composite.

### Example 4 Composite for Relieving Functional Dyspepsia

1 g of dried orange peels, 3 g of figs, and 2.5 g of seville orange flowers were uniformly mixed to obtain a mixture. The obtained mixture was crushed, 65 mL of water was added, the mixture was decocted at 100° C. for 1.5 hours. Then 52 mL of water was added, the mixture was decocted at 100° C. for 1 hour. Then the mixture was filtered by using a 40-mesh gauze, centrifuged at 5,000 rpm for 6 minutes, concentrated at a temperature of 60° C. and a pressure intensity of −0.09 MPa until a brix value was 25%, and dried at a temperature of −45° C. and a pressure intensity of 12 Pa for 5 hours to obtain the composite.

### Example 5 Composite for Relieving Functional Dyspepsia

4 g of dried orange peels, 0.5 g of figs, and 0.5 g of seville orange flowers were uniformly mixed to obtain a mixture. The obtained mixture was crushed, 50 mL of water was added, the mixture was decocted at 100° C. for 1.5 hours. Then 40 mL of water was added, the mixture was decocted at 100° C. for 1 hour. Then the mixture was filtered by using a 40-mesh gauze, centrifuged at 5,000 rpm for 6 minutes, concentrated at a temperature of 60° C. and a pressure intensity of −0.09 MPa until a brix value was 25%, and dried at a temperature of −45° C. and a pressure intensity of 12 Pa for 5 hours to obtain the composite.

### Comparative Example 1

The method is the same as that in example 1. A difference is that only dried orange peels were added without adding figs and seville orange flowers, and a mass of the dried orange peels is 10 g.

### Comparative Example 2

The method is the same as that in example 1. A difference is that only figs were added without adding dried orange peels and seville orange flowers, and a mass of the figs is 10 g.

### Comparative Example 3

The method is the same as that in example 1. A difference is that only seville orange flowers were added without adding figs and dried orange peels, and a mass of the seville orange flowers is 10 g.

### Comparative Example 4

The method is the same as that in example 1. A difference is that the dried orange peels were replaced with exocarpium citrus grandis, and a mass is unchanged.

### Comparative Example 5

The method is the same as that in example 1. A difference is that the figs were replaced with longan aril, and a mass is unchanged.

### Comparative Example 6

The method is the same as that in example 1. A difference is that the seville orange flowers were replaced with fructus aurantii, and a mass is unchanged.

### Experimental Example 1 Composite May Relieve Functional Dyspepsia

### 1. Materials

SD rats come from the Experimental Animal Center of Zhejiang Academy of Medical Sciences, having the production license number of SCXK (Zhe) 2019-0002. 50 DS rats are all male, weigh 150 to 180 g and are divided into 10 rats/group. The rats are adapted to the environment in an SPF animal room for 5 to 7 days, wherein environment conditions of the SPF animal room are as follows: a temperature of 20 to 26° C., a humidity of 40% to 70%, and light and shade alternation time of 12 h/12 h.

The composite for relieving functional dyspepsia (hereinafter referred to as composite) prepared in example 1 was selected and other reagents are domestic reagents.

### 2. Methods

(1) Grouping: the SD rats were randomly divided into a blank group, a model group, a composite low-dose group, a composite medium-dose group, and a composite high-dose group.
(2) Sample administration: the rats in the blank group and the model group were respectively gavaged with 1 mL of normal saline, the rats in the composite low-dose group were administered with 125 mg/kg of the composite, the rats in the composite medium-dose group were administered with 250 mg/kg of the composite, the rats in the composite high-dose group were administered with 500 mg/kg of the composite, and a modeling cycle is 2 weeks.

During the first week, the rats in the five groups were administered 1 time per day and on a free diet. During the second week, the rats in the five groups were still continuously administered 1 time per day and on a free diet, wherein the rats in the model group, the composite low-dose group, the composite medium-dose group, and the composite high-dose group were used to construct a gastric motility disorder model (the SD rats are experimental animals, based on the fact that mental factors are important in pathogenic factors for functional dyspepsia. The method enables the animals to generate dyspepsia symptoms such as delayed gastric emptying, impaired gastric regulatory function, increased visceral sensitivity, and decreased gastric motility by tail clamping. The method has short modeling cycle, high speed, and is favorable for experimental practice operation without obvious organic changes).
(3) Construction of gastric motility disorder model: all the experimental animals of the model group, the composite low-dose group, the composite medium-dose group and the composite high-dose group were molded by tail clamping stimulation and alternate-day feeding.

The tail clamping stimulation is as follows: a far end ⅓ of a tail of a rat was clamped using a long sponge clamp, no skin was broken, the rat was made furious and fought with other rats to irritate the rats in the whole cage; stimulation was performed for 1 time every other 3 hours and for 30 minutes each time (the rat was continuously stimulated within half an hour, along with the aggravation of fighting, the rat may be scratched, and in order to avoid inflammation interference, 0.5% iodophor may be used for applying an injured part to control infection), and the stimulation was performed 4 times a day continuously for 7 days.

The alternate-day feeding is as follows: within 7 days, feeding was not performed in a single day, enough food was fed in even-numbered days, and the rat was on a free diet.
(4) Determination of efficacy of composite: a stomach residue rate and an intestinal propulsive rate were detected. Each group of the experimental animals were fasted for 24 hours before a last sample administration, and gavaged with 0.8 mL/rat of a semisolid paste made of gelatin after 1 hour of the last administration. The animals were sacrificed after 30 minutes to measure the intestinal propulsive rate and the stomach residue rate.

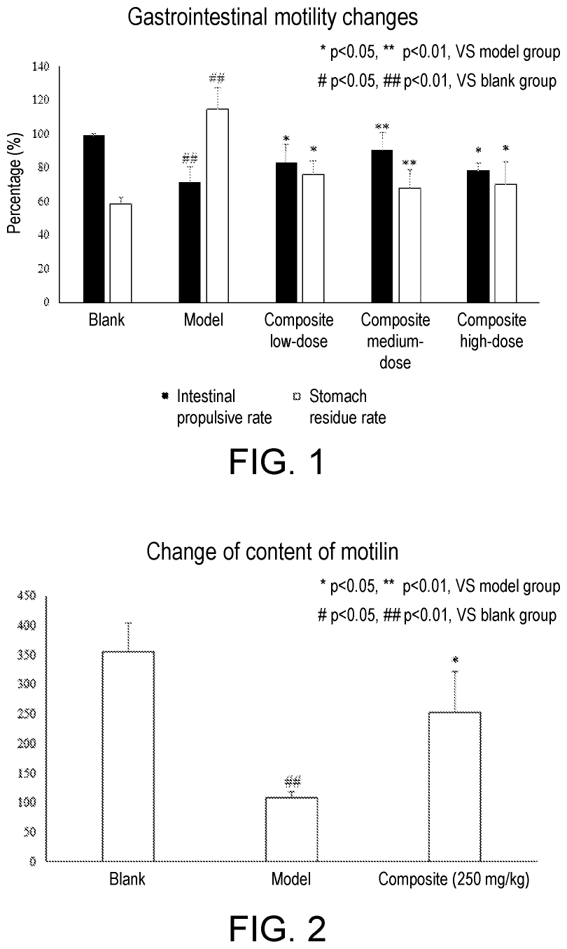

The obtained results of the intestinal propulsive rate and the stomach residue rate were shown in FIG. 1. It can be seen that compared with the blank group, the animals in the model group have an extremely significantly reduced intestinal propulsive rate (p<0.01) and an extremely significantly increased stomach residue rate (p<0.01), which indicates that the modeling is successful. Compared with the model group, the rats in the composite low-dose group, the composite medium-dose group and the composite high-dose group have an obviously improved intestinal propulsive rate (all p<0.05) and an obviously reduced stomach residue rate (all p<0.05), which indicates that the composite of the present invention has obvious functions of promoting gastric emptying and intestinal motility and may effectively relieve functional dyspepsia. Besides, the differences between the composite medium-dose group and the model group are the most obvious and the composite medium-dose group has the best recovery of gastrointestinal motility. Therefore, the following experimental animals all used the composite of 250 mg/kg.
(5) Detection of efficacy mechanism of composite: an efficacy mechanism of the composite was investigated from the perspectives of gastrointestinal hormone secretion, inflammatory factor expression and Cajal interstitial cell viability. After the rats in the blank group, the model group and the composite medium-dose group were administrated for a last time, levels of gastrointestinal hormone motilin (MOT), cholecystokinin (CCK), vasoactive intestinal peptide (VIP), inflammatory cytokine tumor necrosis factor α (TNF-α) and interferon γ (IFN-γ) in serum were detected by ELISA, and an expression level of a Cajal interstitial cell index component c-kit was examined by real-time PCR.

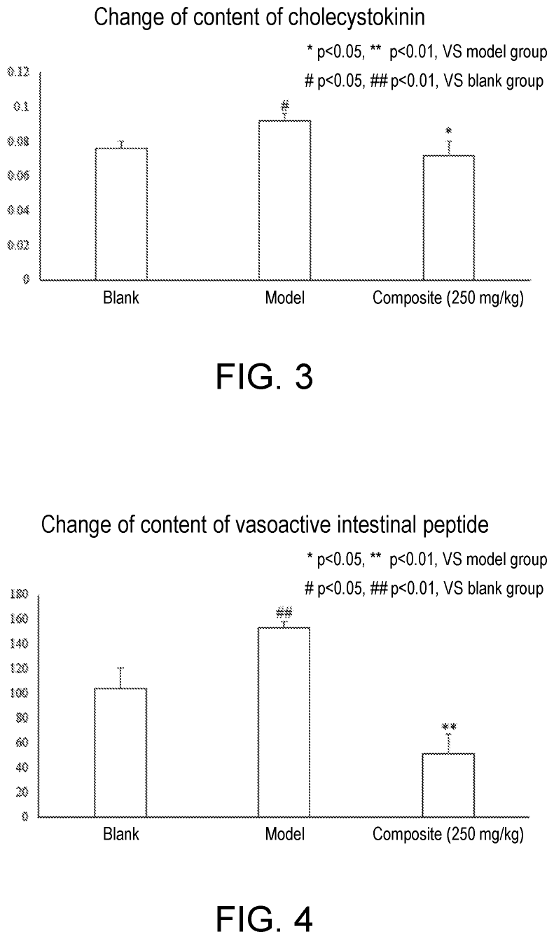

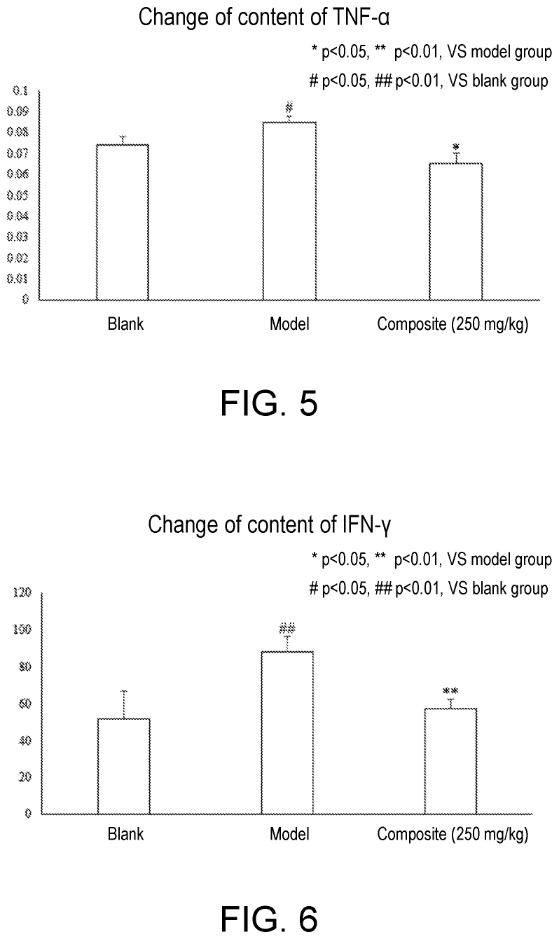

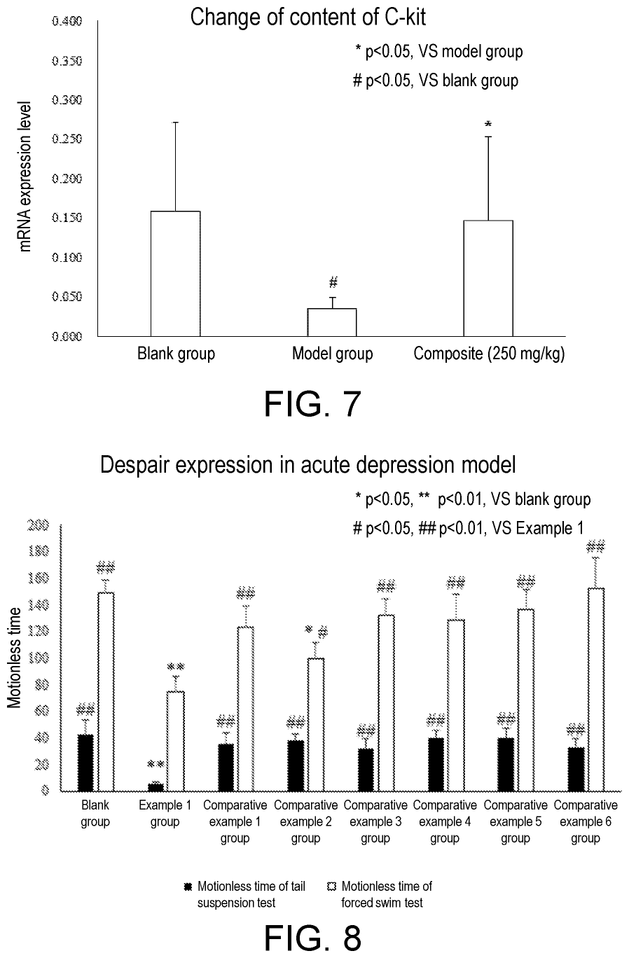

The obtained results are shown in FIGS. 2 to 7. FIG. 2 shows a change of content of motilin, FIG. 3 shows a change of content of cholecystokinin, FIG. 4 shows a change of content of vasoactive intestinal peptide, FIG. 5 shows a change of content of TNF-α, FIG. 6 shows a change of content of IFN-γ, and FIG. 7 shows a change of content of c-kit. As can be seen from the figures:
(1) Compared with the blank group, the model group had a remarkably reduced MOT expression level (p<0.01) and remarkably increased CCK and VIP expression levels (both p<0.05), which indicates that a brain-gut axis function of the animals in the model group was seriously disordered and the gastric motility was remarkably reduced; the expression levels of TNF-α and IFN-γ were obviously increased, which indicates that the gastric mucosa of the animals in the model group had an inflammatory reaction and the gastric emptying was seriously inhibited; and the expression level of c-kit was obviously reduced, which indicates that the activity of a gastrointestinal motility cell, namely a Cajal interstitial cell was obviously reduced and the gastrointestinal motility was seriously disordered. These all indicated successful modeling.
(2) Compared with the model group, the composite medium-dose group may restore normal expressions of MOT, CCK, VIP and c-kit and inhibit levels of inflammatory factors TNF-α and IFN-γ, which indicates that the composite of the present invention has obvious functions of promoting gastrointestinal motility and inhibiting inflammatory reaction and may effectively relieve functional dyspepsia.

The secretion level of the gastrointestinal hormone may reflect the function of the brain-gut axis: MOT may promote gastrointestinal motility and gastric emptying; CCK has effects of delaying gastric emptying, inhibiting ingestion, causing gastric electrical dysrhythmia, promoting gallbladder contraction, increasing pancreatic secretion and inducing satiety; and VIP is an inhibitory neurotransmitter and may relax smooth muscles (reduce gastrointestinal motility), reduce visceral resistance, slow down gastric emptying, and inhibit small intestine movement. A proinflammatory factor reflects a gastrointestinal barrier function: T cells produce an inflammatory factor IFN-γ, mediate activation of neutrophils and macrophages, and participate in inflammatory reaction of gastric mucosa. The chronic inflammatory reaction of the gastric mucosa is closely related to the occurrence of functional dyspepsia. Macroscopically, the composite may promote gastric emptying and small intestine transportation. Microscopically, the composite may promote expressions of MOT and c-kit, inhibits expressions of CCK, VIP, TNF-α and IFN-γ, and further synergistically plays a role in regulating a brain-gut axis function, inhibiting inflammatory reaction and improving activity of gastrointestinal motility cells, wherein the inflammatory reaction can influence the activity of the gastrointestinal motility cells and is reflected on the brain-gut axis function, abnormity of the brain-gut axis function may promote deterioration of the inflammatory reaction and further lead to inactivation of the gastrointestinal motility cells, or the inactivation of the gastrointestinal motility cells can be fed back to the brain-gut axis, such that the function of the gastrointestinal motility cells is abnormal and further leads to/worsens a proinflammatory response.

### Experimental Example 2 Composite May Improve Sleep

1. Principle: a pentobarbital sodium subthreshold dose hypnosis test, a sleep latency test and a sleep extension time change test are common test methods for a sleep improvement performance of a health-care food. The method takes a disappearance of a righting reflex as an observation index and is specifically represented as follows: when a mouse is placed on the back to lie, if the mouse cannot turn over for more than 60 s, the righting reflex is considered to disappear, and the mouse falls sleep; if a body position can be righted immediately (within 5 s), the righting reflex is considered to be recovered and the mouse is in a waking state; and a time from the disappearance of the righting reflex to the recovery is an animal sleeping time.
2. Materials: ICR mice come from the Experimental Animal Center of Zhejiang Academy of Medical Sciences and have the production license number of SCXK (Zhe). The mice are all male, weigh 18 to 22 g and are divided into 10 mice/group. The mice are adapted to the environment in an SPF animal room for 5 to 7 days, wherein environment conditions of the SPF animal room are as follows: a temperature of 20 to 26° C., a humidity of 40% to 70%, and light and shade alternation time of 12 h/12 h.
3. Methods:
(1) Grouping: the ICR mice were divided into a blank group, a positive control group, an example 1 group, an example 2 group, an example 3 group, an example 4 group, an example 5 group, a comparative example 1 group, a comparative example 2 group, a comparative example 3 group, a comparative example 4 group, a comparative example 5 group, and a comparative example 6 group.
(2) Sample administration: all groups of the mice were on a free diet for 7 days. After the mice were adapted to the environment, the mice in the blank group were gavaged with 1 mL of normal saline, the mice in the positive control group were gavaged with 100 mg/kg of y-aminobutyric acid (GABA), the mice in the example 1 to 5 groups were gavaged with 250 mg/kg of the composite obtained in examples 1 to 5, respectively, and the mice in the comparative example 1 to 6 groups were respectively gavaged with 250 mg/kg of the product obtained in comparative examples 1 to 6, and continuously gavaged for 14 days for 1 time every day. The mice were on a free diet during the period. After the gavage was finished, a sleep improvement efficacy test experiment was performed.
(3) Pentobarbital sodium subthreshold dose hypnosis test: firstly, a pre-experiment determined that a pentobarbital sodium subthreshold hypnosis dose (namely the maximum subthreshold dose of pentobarbital sodium where 80% to 90% of a righting reflex of a mouse does not disappear) was 30 mg/kg BW; and after a last administration of normal saline and test samples with different concentrations and 15 min before a peak effect appeared, the maximum subthreshold dose of pentobarbital sodium was injected into an abdominal cavity of each group of animals, the number of the animals falling asleep within 30 min (when the righting reflex disappeared for more than 1 min) was recorded, a sleeping rate was calculated, and the results were shown in Table 1. The experiment was preferably performed in a quiet environment at a temperature of 24° C. to 25° C.
(4) Sleep latency experiment: firstly, a pre-experiment determined that a dose of pentobarbital sodium when 100% animals fall asleep but a sleeping time is not too long (no more than 10 min) was 200 mg/kg BW; and after a last administration of normal saline and test samples with different concentrations for 15 min, pentobarbital sodium was injected into an abdominal cavity of each group of animals at an injection amount of 0.2 mL/20 g, and a disappearance of a righting reflex was used as an index and an influence of the test samples on a sleep latency of pentobarbital sodium was observed.
(5) Sleep extension time change experiment: firstly, a pre-experiment determined that a dose of pentobarbital sodium when 100% animals fall asleep but a sleeping time is not too long was 30 mg/kg BW; and after a last administration of normal saline and test samples with different concentrations and 15 min before a peak effect appeared, pentobarbital sodium was injected into an abdominal cavity of each group of animals at an injection amount of 0.2 mL/20 g, and a disappearance of a righting reflex was used as an index and whether the test samples may prolong sleeping time of pentobarbital sodium was observed.
4. Results:

The results are shown in Table 1.

TABLE 1

| | Sleeping | Sleep latency | Sleeping time |
|---|---|---|---|
| | rate | x̅ ± s | x̅ ± s |
| Groups | (%) | (min) | (min) |
| Blank group | 10.00 | 7.78 ± 2.65 | 30.08 ± 6.42 |
| Positive | 90.00** | 5.60 ± 1.48* | 92.36 ± 16.67** |
| control group | | | |
| Example 1 | 90.00** | 5.64 ± 0.81* | 95.88 ± 13.19** |
| group | | | |
| Example 2 | 70.00* | 5.87 ± 1.37* | 72.90 ± 12.21** |
| group | | | |
| Example 3 | 70.00* | 5.75 ± 0.76* | 74.13 ± 15.45** |
| group | | | |
| Example 4 | 50.00* | 6.29 ± 1.02 | 58.69 ± 17.24* |
| group | | | |
| Example 5 | 40.00 | 6.32 ± 2.20 | 52.81 ± 9.98* |
| group | | | |
| Comparative | 20.00 | 6.66 ± 2.31 | 35.50 ± 8.79 |
| example 1 | | | |
| group | | | |
| Comparative | 10.00 | 7.04 ± 1.89 | 33.44 ± 7.68 |
| example 2 | | | |
| group | | | |
| Comparative | 30.00 | 7.49 ± 2.12 | 41.33 ± 9.35 |
| example 3 | | | |
| group | | | |
| Comparative | 0 | 8.01 ± 3.21 | 32.15 ± 7.34 |
| example 4 | | | |
| group | | | |
| Comparative | 20.00 | 6.97 ± 2.54 | 43.62 ± 5.39 |
| example 5 | | | |
| group | | | |
| Comparative | 0 | 7.57 ± 3.09 | 30.74 ± 7.03 |
| example 6 | | | |
| group | | | |
| Blank group | 10.00 | 7.78 ± 2.65 | 30.08 ± 6.42 |

Note:
compared with the blank group, *p < 0.05 and **p < 0.01.

As can be seen from Table 1:
(1) The mice in the example 1 to 5 groups were remarkably superior to the mice in the blank group in the sleeping rate, sleep latency and sleeping time, which indicated that the composite of the present invention may remarkably increase the sleeping rate (p<0.05) of the mice injected with a subthreshold dose of pentobarbital sodium, shorten the sleep latency of the mice (p<0.05) and prolong the sleeping time (p<0.01) of the mice. Therefore, the composite of the present invention was beneficial to improving sleep and may be used for treating insomnia.
(2) The sleeping rate of the mice in the example 1 group was superior to the sum of the sleeping rates of the mice in the comparative example 1 to 3 groups (prepared from only one raw material in the composite). Besides, based on the blank group, the sleep extension time of the example 1 group was longer than the sum of the extension time of the comparative example 1 to 3 groups, which indicated that the dried orange peels, figs, and seville orange flowers in the composite in the example 1 group played a synergistic effect in improving sleep.
(3) The mice in the example 1 group were remarkably superior to those in the comparative example 4 to 6 groups in the sleeping rate, sleep latency and sleeping time (types of raw materials of the composite were changed), which indicated that since the present invention specifically selected the three ingredients of the dried orange peels, figs, and seville orange flowers as the raw materials for the composite, the composite of the present invention can have such an excellent sleep improvement effect.

### Example 3 Composite May Treat and/or Prevent Depression

1. Principle: effects of the composite in preventing depression were investigated from an open field test, a tail suspension test and a forced swim test. The open field test is a method for evaluating excitation or inhibition of a central nervous system of a mouse through behavioral manifestations and exploratory behaviors of the mouse in a strange environment, such as spontaneous behaviors, motor conditions, emotional states and the like. The suspension test or forced swim test is a method for measuring depression-like behaviors of a mouse by reflecting hopelessness of the mouse after repeated struggle through duration of a motionless state of the mouse under a stress condition.
2. Materials: ICR mice come from the Experimental Animal Center of Zhejiang Academy of Medical Sciences and have the production license number of SCXK (Zhe). The mice are all male, weigh 18 to 22 g and are divided into 10 mice/group. The mice are adapted to the environment in an SPF animal room for 5 to 7 days, wherein environment conditions of the SPF animal room are as follows: a temperature of 20 to 26° C., a humidity of 40% to 70%, and light and shade alternation time of 12 h/12 h.
3. Methods:
(1) Grouping: the ICR mice were divided into a blank group, an example 1 group, a comparative example 1 group, a comparative example 2 group, a comparative example 3 group, a comparative example 4 group, a comparative example 5 group, and a comparative example 6 group.
(2) Sample administration: all groups of the mice were on a free diet for 7 days. After the mice were adapted to the environment, the mice in the blank group and the model group were gavaged with 1 mL of normal saline, the mice in the example 1 group were gavaged with 250 mg/kg of the composite obtained in example 1, and the mice in the comparative example 1-6 groups were respectively gavaged with 250 mg/kg of the product obtained in comparative examples 1 to 6, and continuously gavaged for 28 days for 1 time every day. The mice were on a free diet during the period. After the gavage was finished, an open field test, a tail suspension test and a forced swim test were successively performed.
(3) Open field test: on day 28 of the administration, the open field test was started 1 hour after the gavage. The mice were placed in a test box while faced a wall from an edge and detected after adapted for 3 min, and autonomous movements (total distance, movement distance, movement time and movement speed) of the mice in a 5-minute test period were observed and counted.
(4) Tail suspension test: on day 28 of the administration, after 1 hour of the gavage, a pre-cut adhesive tape was wound at a position 1-1.5 cm away from a tail tip part of a tail of the mouse, the mouse hung on an S-shaped small iron hook to be in an inverted suspension state, a head was kept 5-10 cm away from a bottom surface of a tail suspension box, and after the mouse adapted for 2 min, a detection was performed by a system. At the moment, as long as the mouse started to struggle, a tension on the iron hook changed, after a sent signal was received by a sensor on a bracket of the tail suspension box, the system would automatically record motionless time of the mouse by the system for 4 min, and finally the accumulated motionless time in the detection period (4 min) was counted (the mouse stopped struggling or was in a motionless state).
(5) Forced swim test: on day 28 of the administration, after 1 hour of the gavage, the mice were placed in a constant-temperature swimming instrument (a height of 20 cm, a diameter of 14 cm, a water depth of 15 cm, and a water temperature of 23±1° C.) for adaptation for 2 min. After the adaptation, the accumulated motionless time of the mice in 4 min (the mouse stopped struggling or presented a floating state, and four limbs slightly moved to keep a head motionlessly on a water surface) was immediately recorded and counted by a computer image real-time detection, analysis and processing system.
4. Results:

The results of the open field test were shown in Table 2 and the results of the tail suspension test and the forced swim test were shown in FIG. 8.

TABLE 2

| | Total | Movement | Movement | Movement |
|---|---|---|---|---|
| | distance | distance | time | speed |
| Groups | (cm) | (cm) | (s) | (cm/s) |
| Blank group | 1529.61 ± | 1263.12 ± | 85.46 ± | 19.15 ± |
| | 275.11 | 210.37 | 15.86 | 0.76 |
| Example 1 group | 1725.24 ± | 1388.01 ± | 88.29 ± | 19.75 ± |
| | 246.79 | 285.81 | 13.74 | 0.63 |

As shown in Table 2, in the open field test, compared with the blank group, the total distance, the movement distance, the movement time and the movement speed of the example 1 group had no significant difference, which indicated that the composite of the invention does not have an excitation effect on a central nervous system of the mice and a false positive result of an antidepressant effect of the composite was excluded.

As can be seen from FIG. 8:
(1) In the tail suspension test and the forced swim test, the motionless time of the mice in the example 1 group was significantly shorter than that of the mice in the blank group, which indicated that the composite of the present invention may significantly relieve an acute despair behavior of the mice caused by tail suspension and an acute depression behavior of the mice in the forced swim environment. Therefore, the composite of the present invention may effectively treat and/or prevent depression.
(2) In the tail suspension test and the forced swim test, the motionless time of the mice in the example 1 group was significantly shorter than that of the mice in the comparative example 1 to 3 groups (prepared from only a single raw material in the composite). Besides, the motionless time of the mice in the comparative example 1 to 3 groups was not significantly different from that of the mice in the blank group on a whole (although the motionless time of the mice in the forced swim test in the comparative example 2 was significantly different from that of the mice in the blank group, but the difference was far inferior to that between the example 1 group and the blank group), which indicated that an effect of preventing and treating depression cannot be effectively achieved by the dried orange peels alone, the figs alone, and the seville orange flowers alone. Since the present invention specifically selected the three ingredients of the dried orange peels, figs, and seville orange flowers as the raw materials for the composite, the three ingredients can have a synergistic effect in preventing and treating depression.
(3) In the tail suspension test and the forced swim test, the motionless time of the mice in the example 1 group was significantly shorter than that of the mice in the comparative example 4 to 6 groups (the types of raw materials of the composite were changed), which indicated that since the present invention specifically selected the three ingredients of the dried orange peels, figs, and seville orange flowers as the raw materials for the composite, the composite of the present invention can have such an excellent effect in preventing and/or treating depression.

The above examples are preferred embodiments of the present invention. However, the embodiments of the present invention are not limited by the above examples. Any change, modification, substitution, combination and simplification made without departing from the spiritual essence and principle of the present invention should be an equivalent replacement manner, and all are included in a protection scope of the present invention.

What is claimed is:

1. A composite for relieving functional dyspepsia, comprising following components in parts by weight: 2 to 8 parts of dried orange peels, 1 to 6 parts of figs, and 1 to 5 parts of seville orange flowers.

2. The composite according to claim 1, wherein the composite comprises the following components in parts by weight: 4 to 6 parts of the dried orange peels, 2 to 4 parts of the figs, and 1 to 3 parts of the seville orange flowers.

3. A preparation method for a composite for relieving functional dyspepsia, comprising:
uniformly mixing dried orange peels, figs and seville orange flowers of formula amount to obtain a mixture; and
successively subjecting the obtained mixture to crushing, water extraction, filtration, centrifugation, concentration and freeze-drying, so as to obtain the composite.

4. The preparation method according to claim 3, wherein the water extraction is as follows: performing heating at 95° C. to 100° C. for 2 to 3 hours.

5. The preparation method according to claim 3, wherein the filtration is performed using a 30 to 50-mesh gauze.

6. The preparation method according to claim 3, wherein the centrifugation is performed at 4,000 to 6,000 rpm for 5 to 8 minutes.

7. The preparation method according to claim 3, wherein the concentration is performed at a temperature of 55° C. to 65° C. and a pressure intensity of −0.1 MPa to −0.08 MPa until a brix value is 20% to 30%.

8. The preparation method according to claim 3, wherein the freeze-drying is performed at a temperature of −50° C. to −40° C. and a pressure intensity of 10 to 13 MPa for 4 to 6 hours.

9. A use of the composite according to claim 1 in preparation of a drug, a food and/or a health-care product for treating and/or preventing functional dyspepsia.

10. A use of the composite according to claim 2 in preparation of a drug, a food and/or a health-care product for treating and/or preventing functional dyspepsia.

11. A drug for treating and/or preventing functional dyspepsia, comprising the composite according to claim 1.

12. A food for treating and/or preventing functional dyspepsia, comprising the composite according to claim 1.

13. A health-care product for treating and/or preventing functional dyspepsia, comprising the composite according to claim 1.

14. A drug for treating and/or preventing functional dyspepsia, comprising the composite according to claim 2.

15. A food for treating and/or preventing functional dyspepsia, comprising the composite according to claim 2.

16. A health-care product for treating and/or preventing functional dyspepsia, comprising the composite according to claim 2.
